(19) United States
(12) Patent Application Publication
(10) Pub. No.: US 2013/0071919 A1
(43) Pub. Date: Mar. 21, 2013

(54) METHOD OF SELECTING INDUCED PLURIPOTENT STEM CELL

(71) Applicants: Shinya Yamanaka, Kyoto (JP); Keisuke Okita, Kyoto (JP)

(72) Inventors: Shinya Yamanaka, Kyoto (JP); Keisuke Okita, Kyoto (JP)

(73) Assignee: KYOTO UNIVERSITY, Kyoto-shi, Kyoto (JP)

(21) Appl. No.: 13/583,830

(22) PCT Filed: Feb. 25, 2011
(86) PCT No.: PCT/JP2011/055011

§ 371 Date: Dec. 5, 2012

Related U.S. Application Data
(60) Provisional application No. 61/312,536, filed on Mar. 10, 2010.

(51) Int. Cl. C12Q 1/68

(52) U.S. Cl. CPC C12Q 1/6837
USPC 435/325; 506/9; 506/16

(57) ABSTRACT

The present invention provides a method of selecting a highly safe induced pluripotent stem cell, which includes comprehensively detecting the sequence of an expression vector used for induction of the induced pluripotent stem cell, in the nucleic acid in the cell, and a kit used for the method.

### TECHNICAL FIELD OF THE INVENTION

The present invention relates to a method of selecting a highly safe induced pluripotent stem cell by comprehensively detecting the sequence of an expression vector used for induction of the induced pluripotent stem cell, and a kit to be used for the method.

### BACKGROUND OF THE INVENTION

In recent years, Yamanaka et al. prepared iPS cells by transferring into mouse fibroblasts the Oct3/4, Sox2, Klf4 and c-Myc genes, and forcing the fibroblasts to express the genes (1, 2). Since the iPS cells can be produced by using a cell derived from a patient to be the treatment target, it is expected to be a transplantation material free of rejection.

On the other hand, in a gene therapy of an X-linked severe combined immunodeficiency disease (X-SCID) in 2002 using a retrovirus vector, the death of the patient due to the onset of leukemia has been reported. This was not attributed to the introduced gene but suspected to have been caused by the excess expression of an unexpected endogenous gene by the virus vector non-specifically integrated into the chromosome. For use as a medical material, therefore, it is desired that the expression vector used for gene transfer be not integrated into the chromosome even partially as a fragment.

Thus, various approaches have been reportedly made to intracellularly express a reprogramming factor while preventing integration of a gene into the chromosome, when establishing iPS cells (3, 4). However, even when this method is used, it is not possible to decisively conclude as long as an expression vector is used that the introduced expression vector has not been integrated into the chromosome.

PCR method has heretofore been used for confirmation of an expression vector integrated into the chromosome (3). Using this method, however, it is not possible to confirm integration of a part outside the amplification range detectable by the PCR method, when such part should be fragmented and integrated into the chromosome.

### CITED REFERENCES

1. WO 2007/069666
2. Takahashi, K. and Yamanaka, S., Cell, 126: 663 (2006)
3. Okita K, et al., Science 322, 949 (2008)
4. WO 2009/133971

### SUMMARY OF THE INVENTION

An object of the present invention is to select an induced pluripotent stem cell (iPS cell) without an expression vector used for the induction remaining in the cell. Therefore, the problem of the present invention is to provide a method of comprehensively examining whether an expression vector used for the induction is contained, even a part thereof, in the iPS cell, and a kit to be used for the method.

To solve the above-mentioned problem, the present inventors have verified the presence of the sequence of an expression vector in iPS cells using a tiling array containing a probe consisting of the sequence of the expression vector for the purpose of examining the presence or otherwise, in the DNA extracted from the iPS cells, of even a part of the sequence of the expression vector used for the induction of the iPS cells. As a result, the intracellular presence of the expression vector used for the induction was confirmed in the iPS cells known to have an expression vector integrated in the chromosome. Moreover, it was confirmed that iPS cells considered to have no expression vector in the cell thereof according to the conventional methods did not contain a fragmented expression vector even partially in the cells.

From the above results, the present inventors have found that whether or not the expression vector used for the to induction is contained in the iPS cells can be comprehensively determined by using the sequence of the vector, which resulted in the completion of the present invention.

Accordingly, the present invention provides the following.

[1] A method of examining an induced pluripotent stem cell comprising a step of comprehensively detecting whether a nucleic acid in an induced pluripotent stem cell established from a somatic cell contains at least one sequence of an expression vector used for inducing pluripotent stem cell other than a sequence inherently present in the genome of the somatic cell.
[2] The method of [1], further comprising a step of selecting an induced pluripotent stem cell in which the sequence of the expression vector is not detected in the detecting step.
[3] The method of [1] or [2], wherein the aforementioned detecting step is performed using a microarray containing a probe comprising a part of the sequence of the expression vector.
[4] The method of [3], wherein the aforementioned microarray is a tiling array.
[5] The method of [3], wherein the aforementioned probe consists of a part of the sequence in the expression vector, which sequence is inherently absent in the genome of the original somatic cell.
[6] The method of [1] or [2], wherein the aforementioned nucleic acid is a chromosomal DNA of an induced pluripotent stem cell.
[7] The method of any of [1] to [6], wherein the aforementioned expression vector is a plasmid.
[8] A microarray comprising a probe consisting of a part of the sequence of an expression vector used for inducing an induced pluripotent stem cell from a somatic cell, wherein the probe can comprehensively detect at least a sequence, from among the sequences of the expression vector, inherently absent in the genome of the somatic cell.
[9] The microarray of [8], wherein the aforementioned probe consists of a part of the sequence in the expression vector, which sequence is inherently absent in the genome of the original somatic cell.
[10] A kit for selecting an induced pluripotent stem cell derived using the aforementioned expression vector, which comprises the microarray of [8] or [9].
[11] The kit of [10], further comprising a package insert stating that the aforementioned microarray can or should be used for selecting an induced pluripotent stem cell.
[12] An induced pluripotent stem cell selected by the method of any of [2] to [7].

Using the present invention, a highly safe iPS cell can be selected, which is free of even a partial integration of the expression vector used for the induction into the cell. Therefore, it is extremely useful for application of an iPS cell to the regenerative medicine.

### BRIEF DESCRIPTION OF THE DRAWINGS

FIG. 1 shows schematic view of two kinds of expression vectors used for preparing iPS cells, wherein the left drawing shows the first expression vector as a whole, A is a pCX backbone of the expression vector not encoded with a reprogramming substance, B in the left drawing is a part where sequences encoding reprogramming substances (Oct3/4, Klf4 and Sox2) are linked by 2A sequence. In the right drawing, C shows a part of the second expression vector, where c-Myc is encoded. The rest of the expression vector is the pCX backbone of A.

FIG. 2 shows the results of tiling array analysis. A to C correspond to each part of the expression vector of FIG. 1. Respective panels show the results of genome DNA derived from fetal mouse fibroblast (MEF origin), which was used for the induction of iPS cells, genome DNAs derived from each iPS cell line (440A-3, 440A-1) and genome DNA derived from 440A-3 added with two kinds of expression vectors shown in FIG. 1 to achieve one copy per one cell (440A-3+plasmid). The horizontal axis of the data shows functional sequences (arrow) in an expression vector shown under each panel and a sequence (spacer) therebetween, and the vertical axis shows the amount of array fragment at said position.

### DETAILED DESCRIPTION OF THE INVENTION

The present invention provides a method for selecting an iPS cell, comprising a step of comprehensively detecting, in the nucleic acid in an induced pluripotent stem cell, the sequence of an expression vector used for the induction of an induced pluripotent stem cell, and a step of selecting an induced pluripotent stem cell in which the sequence of the expression vector is not detected in the chromosome.

The detail of each step and the kit used for the method of the present invention are shown below.

### I. Production of iPS Cells

An iPS cell is an artificial stem cell derived from somatic cell, which has nearly the same characteristics as those of ES cells, for example, differentiation pluripotency and the potential for proliferation by self-renewal, and that can be prepared by transferring a certain nuclear reprogramming substance, in the form of nucleic acid or protein, to a somatic cell [K. Takahashi and S. Yamanaka (2006) Cell, 126: 663-676; K. Takahashi et al. (2007) Cell, 131: 861-872; J. Yu et al. (2007) Science, 318: 1917-1920; M. Nakagawa et al. (2008) Nat. Biotechnol., 26: 101-106; WO 2007/069666]. In the present invention, at least one nuclear reprogramming substance is an iPS cell obtained by introduction into a somatic cell in the form of a nucleic acid.

The nuclear reprogramming substance may be any gene specifically expressed in ES cells, or a gene that plays a key role in the maintenance of the undifferentiated state of ES cells, or a gene product thereof. Examples include Oct3/4, Klf4, Klf1, Klf2, Klf5, Sox2, Sox1, Sox3, Sox15, Sox17, Sox18, c-Myc, L-Myc, N-Myc, TERT, SV40 Large T antigen, HPV16 E6, HPV16 E7, Bmi1, Lin28, Lin28b, Nanog, Esrrb and Esrrg. These reprogramming substances may be used in combination when establishing iPS cells. For example, a combination comprising at least one, two or three of these reprogramming substances may be used, with preference given to a combination comprising four.

Information on the nucleotide sequences of the mouse and human cDNAs of the above-described nuclear reprogramming substances is available with reference to the NCBI accession numbers shown in WO 2007/069666. Information on the mouse and human cDNA sequences of L-Myc, Lin28, Lin28b, Esrrb and Esrrg is available with reference to the NCBI accession numbers shown below. Those skilled in the art are able to prepare a desired nuclear reprogramming substance by a conventional method on the basis of the information on the cDNA sequence or amino acid sequence thereof.

| Name of gene | Mouse | Human |
|---|---|---|
| L-Myc | NM_008506 | NM_001033081 |
| Lin28 | NM_145833 | NM_024674 |
| Lin28b | NM_001031772 | NM_001004317 |
| Esrrb | NM_011934 | NM_004452 |
| Esrrg | NM_011935 | NM_001438 |

When these nuclear reprogramming substances are introduced into a somatic cell in the form of a nucleic acid, an expression vector may be used. Examples of the expression vector in the present invention include plasmid, artificial chromosome vector, and virus vector. Examples of the artificial chromosome vector include human artificial chromosome (HAC), yeast artificial chromosome (YAC), bacterial artificial chromosome (BAC, PAC) and the like. In addition, examples of viral vectors include retrovirus vectors, lentivirus vectors (both Cell, 126, pp. 663-676, 2006; Cell, 131, pp. 861-872, 2007; Science, 318, pp. 1917-1920, 2007), adenovirus vectors (Science, 322, 945-949, 2008), adeno-associated virus vectors, Sendai virus vectors (Proc. Jpn. Acad. Ser. B. Phys. Biol. Sci. 85, 348-62, 2009) and the like. Furthermore, plasmids for mammalian cells can be used (Science, 322:949-953, 2008 and WO 2009/032456). In the present invention, when the expression vector is a plasmid, artificial chromosome vector and the like, it can be introduced into a somatic cell according to a method such as lipofection, liposome, microinjection, particle gun method and the like, and when the expression vector is a virus vector, it can be introduced into a somatic cell by infection. The expression vector can contain a regulatory sequence such as a promoter, enhancer, internal ribosomal entry site (IRES), terminator, or polyadenylation site to allow a nuclear reprogramming substance to be expressed.

As the promoter to be used, EF1α promoter, CAG promoter, SRα promoter, SV40 promoter, LTR promoter, CMV (cytomegalovirus) promoter, RSV (Rous sarcoma virus) promoter, MoMuLV (Moloney murine leukemia virus) LTR, HSV-TK (herpes simplex virus thymidine kinase) promoter and the like are used. Of these, EF1α promoter, CAG promoter, MoMuLV LTR, CMV promoter, SRα promoter and the like can be particularly recited.

The expression vector can further contain, as required, a drug resistance gene (e.g., kanamycin resistance gene, ampicillin resistance gene, puromycin resistance gene and the like), a selection marker sequence such as the thymidine kinase gene or diphtheria toxin gene, a reporter gene sequence such as of green fluorescent protein (GFP), β glucuronidase (GUS) or FLAG, and the like. The expression vector may have a loxP sequence placed at both ends of the gene that encodes the nuclear reprogramming substance or of a promoter and the gene connected thereto, to enable resection thereof, after being transferred to somatic cells. In another preferable embodiment, a method including incorporating an introduced gene into a chromosome using transposon, allowing the transferase to act on a cell by using a plasmid vector or an adenovirus vector, and completely removing the introduced gene from the chromosome can be used. Preferable examples of the transposon include piggyback, which is a transposon derived from lepidopterous insect, and the like (Kaji, K. et al., Nature, 458: 771-775 (2009), Woltjen et al., Nature, 458: 766-770 (2009), WO 2010/012077). The vector may also contain the origin and the sequence relating to the replication of lymphotrophic herpes virus, BK virus and bovine papillomavirus to allow the vector to be replicated and occur episomally even without being incorporated in the chromosome. For example, EBNA-1 and oriP or Large T and SV40ori sequence can be contained (WO 2009/115295, WO 2009/157201 and WO 2009/149233). For simultaneous introduction of plural nuclear reprogramming substances, moreover, an expression vector that allows polycistronic expression may be used. For polycistronic expression, sequences encoding a gene may be linked by IRES or foot and mouth disease virus (FMDV) 2A coding region (Science, 322:949-953, 2008; WO 2009/092042 and 2009/152529).

When a part of nuclear reprogramming substances is introduced in the form of a protein, it may be introduced into a somatic cell, for example, by lipofection, conjugation with cellular membrane permeable peptide, microinjection and the like.

To increase iPS cell induction efficiency in nuclear reprogramming, in addition to the above-described factors, for example, histone deacetylase (HDAC) inhibitors [e.g., low-molecular inhibitors such as valproic acid (VPA) (Nat. Biotechnol., 26(7): 795-797 (2008)], trichostatin A, sodium butyrate, MC 1293, and M344, nucleic acid-based expression inhibitors such as siRNAs and shRNAs against HDAC (e.g., HDAC1 siRNA Smartpool® (Millipore), HuSH 29 mer shRNA constructs against HDAC1 (OriGene) and the like), and the like], DNA methyltransferase inhibitors (e.g., 5′-azacytidine) [Nat. Biotechnol., 26(7): 795-797 (2008)], G9a histone methyltransferase inhibitors [e.g., low-molecular inhibitors such as BIX-01294 (Cell Stem Cell, 2: 525-528 (2008)], nucleic acid-based expression inhibitors such as siRNAs and shRNAs against G9a [e.g., G9a siRNA (human) (Santa Cruz Biotechnology) and the like) and the like], L-channel calcium agonists (e.g., Bayk8644) [Cell Stem Cell, 3, 568-574 (2008)], p53 inhibitors [e.g., siRNA and shRNA against p53 (Cell Stem Cell, 3, 475-479 (2008)), Wnt Signaling activator (e.g., soluble Wnt3a) [Cell Stem Cell, 3, 132-135 (2008)], cytokines such as LIF, bFGF etc., ALK5 inhibitors (e.g., SB431542) [Nat Methods, 6: 805-8 (2009)], a mitogen-activated protein kinase signaling inhibitor, a glycogen synthase kinase-3 inhibitor [PloS Biology, 6(10), 2237-2247 (2008)], miRNAs such as miR-291-3p, miR-294, and miR-295 [R. L. Judson et al., Nat. Biotechnol., 27:459-461 (2009)], and the like can be used.

Examples of culture media for iPS cell induction include (1) a DMEM, DMEM/F12 or DME medium containing 10 to 15% FBS (these media can further contain LIF, penicillin/streptomycin, puromycin, L-glutamine, non-essential amino acids, (β-mercaptoethanol and the like), (2) an ES cell culture medium containing bFGF or SCF, for example, a mouse ES cell culture medium (e.g., TX-WES medium, Thromb-X NV) or a primate ES cell culture medium [e.g., primate (human and monkey) ES cell culture medium, ReproCELL, Kyoto, Japan], and the like. In this case, a low protein medium or cell cycle inhibitor-containing medium may be used for enhancing induction efficiency of IFS cell (WO 2010/004989).

In a culture method, for example, somatic cells and a nuclear reprogramming substance (nucleic acid or protein) are brought into contact with each other on a DMEM or DMEM/F12 medium containing 10% FBS and cultured at 37° C. in the presence of 5% CO₂ for about 4 to about 7 days, after which the cells are re-seeded onto feeder cells (e.g., STO cells, SNL cells and other cells, previously treated with mitomycin C), and again cultured using a bFGF-containing primate ES cell culture medium, starting about 10 days after contact of the somatic cells and the nuclear reprogramming substance, whereby iPS-like colonies can be produced in about 30 to about 45 days or more after the contact. To increase the efficiency of iPS cell induction, the somatic cells may be cultured under conditions involving a low oxygen concentration of 5-10% (WO 2010/013845).

Alternatively, the cells may be cultured on feeder cells (e.g., STO cells, SNL cells and other cells, previously treated with mitomycin C), using a DMEM medium containing 10% FBS (this can further contain LIF, penicillin/streptomycin, puromycin, L-glutamine, non-essential amino acids, β-mercaptoethanol and the like), whereby ES-like colonies can be produced after about 25 to about 30 days or more.

During the period of cultivation, the medium is replaced with a fresh supply of the same medium once daily starting on day 2 of cultivation. Although the number of somatic cells used for nuclear reprogramming is not subject to limitations, it falls in the range of about 5×10³ to about 5×10⁶ cells per 100 cm² of culture dish.

When a drug resistance gene is used as a marker gene, cells that express the marker gene can be selected by is cultivation using a medium containing the corresponding drug (selection medium). Cells that express the marker gene can be detected by making an observation using a fluorescence microscope for a fluorescent protein gene as the marker gene, by adding a luminescent substrate for a luminescent enzyme gene as the marker gene, and by adding a color developing substrate for a color developing enzyme gene as the marker gene.

Any cells, other than germ cells, of mammalian origin (e.g., humans, mice, monkeys, pigs, rats and the like) can be used as the “somatic cells” used in the present invention. Examples include keratinizing epithelial cells (e.g., keratinized epidermal cells), mucosal epithelial cells (e.g., epithelial cells of the superficial layer of tongue), exocrine gland epithelial cells (e.g., mammary gland cells), hormone-secreting cells (e.g., adrenomedullary cells), cells for metabolism or storage (e.g., liver cells), intimal epithelial cells constituting interfaces (e.g., type I alveolar cells), intimal epithelial cells of the obturator canal (e.g., vascular endothelial cells), cells having cilia with transporting capability (e.g., airway epithelial cells), cells for extracellular matrix secretion (e.g., fibroblasts), constrictive cells (e.g., smooth muscle cells), cells of the blood and the immune system (e.g., T lymphocytes), sense-related cells (e.g., rod cells), autonomic neurons (e.g., cholinergic neurons), sustentacular cells of sensory organs and peripheral neurons (e.g., satellite cells), neurons and glia cells in the central nervous system (e.g., astroglia cells), pigment cells (e.g., retinal pigment epithelial cells), progenitor cells (tissue progenitor cells) thereof and the like. There is no limitation on the degree of cell differentiation, the age of the animal from which cells are collected and the like; even undifferentiated progenitor cells (including somatic stem cells) and finally differentiated mature cells can be used alike as sources of somatic cells in the present invention. Here, examples of undifferentiated progenitor cells include tissue stem cells (somatic stem cells) such as neural stem cells, hematopoietic stem cells, mesenchymal stem cells, and dental pulp stem cells.

In the present invention, the choice of mammalian individual from which somatic cells are collected is not particularly limited, but it is preferably a human.

### II. Method for Comprehensive Detection of Sequence of Expression Vector in Chromosome

In the present invention, comprehensive detection means detection of all without exception. Detailedly, it means detection at intervals of 25 or less base sequence in the sequence of a desired part of an expression vector, and preferably means detection of the whole sequence of the desired part of an expression vector, without leaving a gap.

In the nucleic acid in an iPS cell produced by the aforementioned method, as a nucleic acid-containing sample for comprehensive detection of the sequence of an expression vector used for inducing the iPS cell, for example, a cell lysate obtained by lysing the iPS cell can be used. While the method for lysing cells is not particularly limited, for example, a method including lysing cellular membrane with an organic solvent such as phenol/chloroform and the like, an alkaline solution, a solution containing a conventionally-known protein denaturant such as sodium iodide, urea, SDS and the like, and a method including mechanically disrupting a cellular membrane by using ultrasonication and the like can be mentioned. In the obtained cell lysate, intracellular nucleases are desirably inactivated. In addition, the sample may contain other substance as long as it does not inhibit hybridization. Preferably, a DNA solution obtained by purifying DNA from a cell lysate and dissolving the DNA in water or a suitable buffer (e.g., TE buffer etc.). More preferably, to detect integration of the expression vector used for inducing iPS cell into a chromosome, a chromosomal DNA solution obtained by extracting only a chromosomal DNA from a cell lysate, and dissolving the DNA in water or a suitable buffer (e.g., TE buffer etc.) is desirable.

Examples of the method for comprehensively detecting the sequence of an intracellular expression vector include Southern blotting method, PCR method, realtime PCR method, microarray method and the like. Preferred is the microarray method, more preferred is the tiling array method.

In the present invention, the tiling array method uses a DNA microarray (DNA chip) wherein a detection probe having a base sequence extracted is immobilized on a substrate in a tile form (generally at regular intervals), based on the sequence information of the expression vector used for inducing the iPS cell.

In the present invention, the constitution of the tiling array can be a general one except that a probe designed to target, of all sequences of the expression vector used for inducing iPS cell from a somatic cell, at least a sequence absent in the genome of the somatic cell is designed such that the gap between probes is not more than 25 bases on average, or the overlap between probes is not more than 99% of the probe length on average. When the overlap between probes is longer, the sequence of the expression vector can be confirmed more elaborately. When only one base does not overlap between probes, the sequence of the expression vector used for induction can be detected by a base unit.

In the present invention, of the sequences of the expression vector, a sequence inherently absent in the genome of the original somatic cell is typically a sequence that does not encode a reprogramming substance or a part thereof (hereinafter to be also referred to as a backbone sequence). For example, promoter, enhancer, IRES, terminator, polyadenylation site, LoxP sequence, replication origin sequences and sequences relating to the replication such as EBNA-1 and oriP or Large T and SV40ori sequences, 2A sequence, a replication origin sequence to amplify an expression vector in Escherichia coli and the like, and a sequences relating to the replication thereof, a selection marker sequence such as a drug resistance gene (e.g., kanamycin resistance gene, ampicillin resistance gene, puromycin resistance gene and the like), thymidine kinase gene, diphtheria toxin gene and the like, a reporter gene sequence such as of green fluorescent protein (GFP), β glucuronidase (GUS), FLAG, and the like, virus structural protein, protease, reverse transcriptase, integrase, envelope, LTR containing elements such as enhancer, promoter, polyadenylation signal etc., and the like in a virus vector, and a meaningless sequence (spacer) to link these functional sequences can be mentioned. However, when a promoter, enhancer, terminator, polyadenylation site etc. of an expression vector contains a sequence inherently present in the original somatic cell, the above does not apply.

The length of the probe can be selected in consideration of the efficiency of signal detection after hybridization and the like. It is generally 20-100 bases, preferably 40-80 bases, more preferably about 60 bases.

When probes are designed by placing a gap between the probes, the gap between the probes is not more than 25 bases, preferably not more than 10 bases, more preferably 0 base, on average. When the probes are designed to overlap, the overlap between the probes is, for example, not more than 10%, not more than 20%, not more than 30%, not more than 40%, not more than 50%, not more than 60%, not more than 70%, not more than 80%, not more than 90%, not more than 95%, or not more than 99%, of the probe length on average. For example, when the probe length is 60 bases, the overlap is 5 bases, 10 bases, 20 bases and the like, more preferably 59 bases. The gap and overlap may be absent and, in this case, the gap is 0 base, or the overlap is 0%.

The probe is not particularly limited as long as it is a nucleic acid containing a base sequence capable of hybridizing, under hybridization conditions usable for general gene expression analysis, with a sequence of the expression vector (which may be a sense strand sequence or an antisense strand sequence) used for induction of iPS cells. Preferably, the probe is a nucleic acid containing a base sequence capable of hybridizing, under stringent conditions, with a sequence of the expression vector used for induction of iPS cells. The “stringent conditions” means conditions under which only a base sequence having identity of not less than 95%, preferably not less than 96%, more preferably not less than 97%, particularly preferably not less than 98%, most preferably not less than 99%, with a completely complementary base sequence to an object nucleic acid sequence can hybridize. Those skilled in the art can easily regulate the conditions to obtain a desired stringency by appropriately changing the salt concentration of hybridization solution, hybridization reaction temperature, probe concentration, probe length, number of mismatches, hybridization reaction time, salt concentration of washing solution, washing temperature, and the like.

These probes can also be obtained by chemically synthesizing using a commercially available DNA/RNA automatic synthesizer and the like. In addition, a chip (array) having a solid-phased probe can be produced by directly synthesizing a probe in situ (on chip) on a solid phase such as silicon, glass and the like.

Such probe can be provided as a solid in a dry state or alcohol precipitated state, and can also be provided in a state of being dissolved in water or a suitable buffer (e.g., TE buffer etc.). When it is used as a labeling probe, the probe can be provided after labeling with any of the following labeling substances in advance, or can be separately provided from labeling substances and can be used by labeling when in use.

As the labeling substance, for example, a radioisotope, an enzyme, a fluorescent substance, a luminescent substance and the like can be used. As the radioisotope, for example, [³²P], [³H], [¹⁴C] and the like can be used. As the above-described enzyme, those that are stable and high in specific activity are preferred; for example, β-galactosidase, β-glucosidase, alkaline phosphatase, peroxidase, malate dehydrogenase and the like can be used. As the fluorescent substance, for example, carbocyanine derivative (e.g., Cy3, Cy5), fluorescein, fluorescamine, fluorescein isothiocyanate, rhodamine, phycoerythrin, allophycocyanin and the like can be used. As the luminescent substance, for example, luminol, luminol derivative, luciferin, lucigenin and the like can be used. Furthermore, a biotin-(strepto)avidin system can also be used for binding of a probe and a labeling agent. On the other hand, when a probe is immobilized on a solid phase, the nucleic acid in a sample can be labeled with a labeling agent similar to the above.

In a preferable embodiment of the present invention, the probe of the present invention is provided in the form of a microarray immobilized on a substrate.

Examples of the material of the substrate include semiconductors such as silicon and the like, inorganic substances such as glass, diamond and the like, films comprising a polymer substance such as poly(ethylene terephthalate), polypropylene and the like as a main component, and the like. In addition, the form of the substrate is not limited, and slide glass, microwell plate, microbeads, fiber type and the like can be mentioned.

Examples of the means to immobilize the probe on a substrate include, but are not limited to, a method comprising introducing a functional group such as amino group, aldehyde group, SH group, biotin and the like into a nucleic acid in advance, introducing a functional group (e.g., aldehyde group, amino group, SH group, streptavidin and the like) that can react with the nucleic acid, on a solid phase, and crosslinking the solid phase and the nucleic acid by a covalent bond between both functional groups, or coating a solid phase with a polycation and immobilizing a polyanionic nucleic acid by utilizing an electrostatic bond, and the like. Examples of the preparation method of microarray include the Affymetrix type wherein a nucleic acid probe is synthesized by a photolithography method synthesizing nucleotide one by one on a substrate (glass, silicon and the like), and the Stanford type wherein a nucleic acid probe prepared in advance is spotted onto a substrate by a microspotting method, an inkjet method, a bubble jet (registered trademark) method and the like. When a probe of 30 mer or more is used, the Stanford type or a combination of the two types is preferable.

By mixing the aforementioned probe and a sample, and detecting the amount of hybridization of the DNA contained in the sample and the probe, the presence of a DNA sequence having a sequence of the probe in the sample can be confirmed. The amount of hybridization can be detected by a method known per se, and can be detected, for example, by the amount of a labeling substance in the labeled probe or in the nucleic acid in a sample.

By detecting, in the nucleic acid in the iPS cell, the sequence of the expression vector used for inducing the iPS cell according to the above-mentioned method, an iPS cell not integrating the expression vector can be selected. For selection, an iPS cell not detected for any sequence of the expression vector used for induction, or a sequence of the expression vector, which is inherently absent in the genome of the original somatic cell (e.g., backbone sequence), is desirably selected.

In the present invention, not detected means a value equivalent or lower than the value detected in any cell known to not contain a fragment of a nucleic acid (e.g., somatic cell used for the production of iPS cell or iPS cell, though not limited to these).

III. Kit for Selection of iPS Cell without Integration of Expression Vector Used for Induction into Chromosome

A kit for comprehensively detecting a sequence of the expression vector for selection of iPS cell in the present invention contains a microarray comprising a probe consisting of a part of the sequence of the aforementioned expression vector.

The kit of the present invention may contain a discrimination analysis means, for example, a written description and instruction of discrimination analysis procedures, a program to perform discrimination analysis procedures using a computer, a program list thereof, a computer-readable recording medium (e.g., flexible disc, optical disc, CD-ROM, CD-R, CD-RW etc.) containing the program, and an apparatus or system to perform the discrimination analysis (computer etc.).

The present invention is explained in more detail in the following by referring to Examples, which are not to be construed as limitative.

### Examples

### Cell

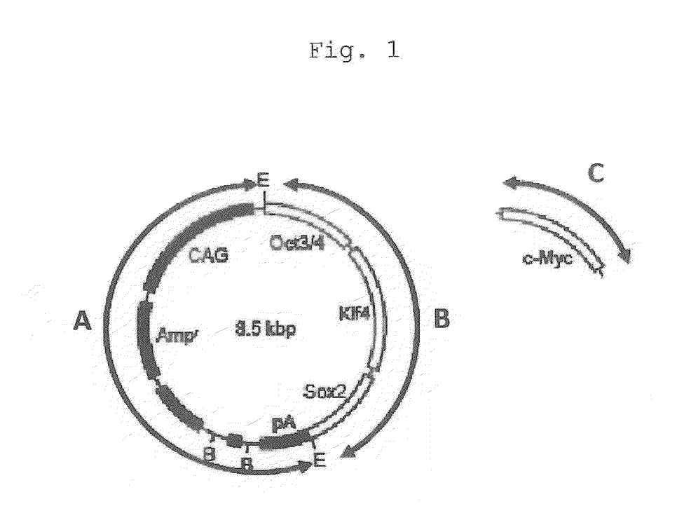

iPS cells produced by the method described in Okita K, et al., Science 322, 949, 2008 (440A-1 and 440A-3) were used. In brief, the cells were iPS cells obtained by introducing pCX-OKS-2A (8495 bp) and pCX-cMyc (6131 bp) (FIG. 1) 4 times every other day into fibroblast derived from a reporter mouse introduced with GFP controlled by Nanog promoter, and confirming expression of the GFP. Here, it was already confirmed that in 440A-1, plasmid was integrated into the chromosome, but otherwise for 440A-3.

### Tiling Array

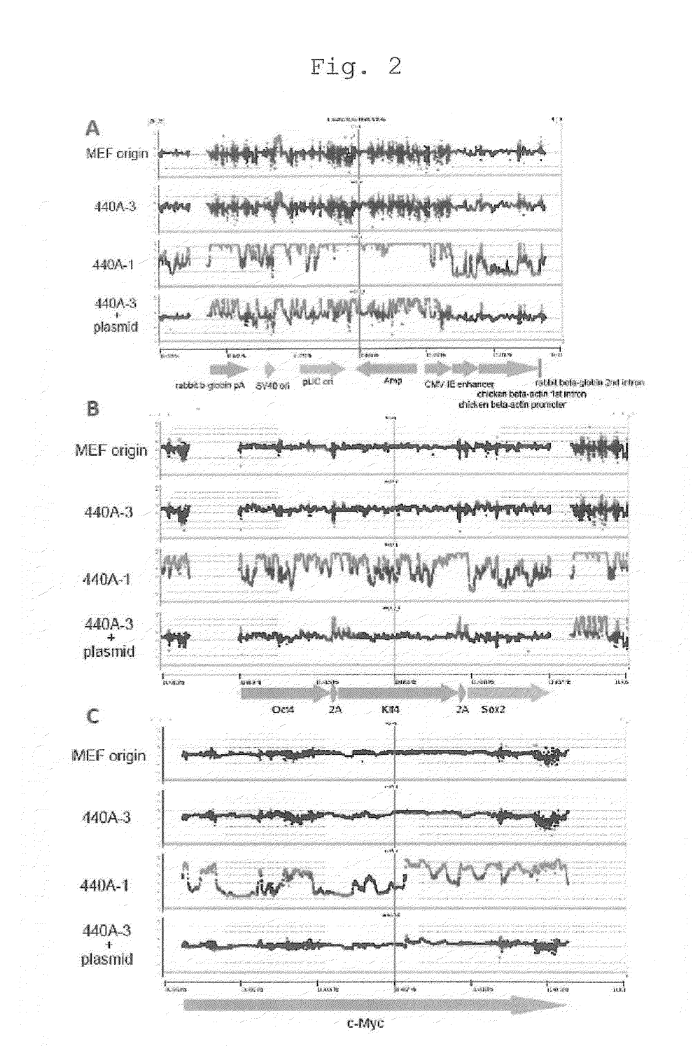

Genomic DNA was extracted from MEF (mouse embryonic fibroblast), 440A-3 and 440A-1 according to a conventional method. The content of each probe sequence in four kinds of DNA solutions of (1) genomic DNA solution of MEF (1.5 μg: corresponding to genomic DNA of about 2.8×10⁵ cells), (2) genomic DNA solution of 440A-3 (1.5 μg), (3) genomic DNA solution of 440A-1 (1.5 μg) and (4) genomic DNA solution of 440A-3 (1.5 μg), added with pCX-OKS-2A and pCX-cMyc by one copy each (pCX-OKS-2A: 2.5 μg: corresponding to about 2.8×10⁵ copy, pCX-cMyc: 1.8 μg: corresponding to about 2.8×10⁵ copy) per one cell was measured by using a Stanford type microarray of probes (pCX backbone (FIG. 1A site): 4736, OKS-2A (FIG. 1B site): 3759, c-Myc (FIG. 1C site): 1395) prepared from sequences obtained by displacing 60 by length each of the backbone plasmid part pCX (FIG. 1A) and DNA sequences of gene regions of pCX-OKS-2A and pCX-cMyc (FIGS. 1B and 1C) by one base. The relative content of each probe in the regions of FIG. 1, A to C, contained in DNAs (1) to (4) is shown in FIG. 2.

As a result, (3) and (4) were confirmed to contain 2A, rabbit-β-globin pA sequence, SV40 on sequence, pUC on sequence, Ampicillin resistance gene sequence and CMV IE enhancer sequence. In addition, (3) was confirmed to contain gene sequences of Oct3/4, Sox2, Klf4 and c-Myc.

The foregoing shows that, in the sequence of a plasmid used to establish an iPS cell, at least the parts other than the gene sequence inherently present in the genome of the cell can be detected, by using Tiling array, from one copy even when a fragment thereof is partially integrated in the chromosome in the iPS cell establishment process.

This application is based on U.S. provisional patent application No. 61/312,536 filed on Mar. 10, 2010, the contents of which are hereby incorporated by reference.

1. A method of examining an induced pluripotent stem cell comprising a step of comprehensively detecting whether a nucleic acid in an induced pluripotent stem cell established from a somatic cell contains at least one sequence of an expression vector used for inducing pluripotent stem cell other than a sequence inherently present in the genome of the somatic cell.

2. The method according to claim 1, further comprising a step of selecting an induced pluripotent stem cell in which the sequence of the expression vector is not detected in the detecting step.

3. The method according to claim 1, wherein the aforementioned detecting step is performed using a microarray containing a probe comprising a part of the sequence of the expression vector.

4. The method according to claim 3, wherein the aforementioned microarray is a tiling array.

5. The method according to claim 3, wherein the aforementioned probe consists of a part of the sequence in the expression vector, which sequence is inherently absent in the genome of the original somatic cell.

6. The method according to claim 1, wherein the aforementioned nucleic acid is a chromosomal DNA of an induced pluripotent stem cell.

7. The method according to claim 1, wherein the aforementioned expression vector is a plasmid.

8. A microarray comprising a probe consisting of a part of the sequence of an expression vector used for inducing an induced pluripotent stem cell from a somatic cell, wherein the probe can comprehensively detect at least a sequence, from among the sequences of the expression vector, inherently absent in the genome of the somatic cell.

9. The microarray according to claim 8, wherein the aforementioned probe consists of a part of the sequence in the expression vector, which sequence is inherently absent in the genome of the original somatic cell.

10. A kit for selecting an induced pluripotent stem cell derived using the aforementioned expression vector, which comprises the microarray according to claim 8.

11. The kit according to claim 10, further comprising a package insert stating that the aforementioned microarray can or should be used for selecting an induced pluripotent stem cell.

12. An induced pluripotent stem cell selected by the method according claim 2.
